(19) United States
(12) Patent Application Publication
(10) Pub. No.: US 2024/0424223 A1
(43) Pub. Date: Dec. 26, 2024

(54) Impact Activated Brake Feature for Drug Delivery Device

(71) Applicant: AMGEN INC., Thousand Oaks, CA (US)

(72) Inventors: Emil Gram Spork, Thousand Oaks, CA (US); Matias Melander, Copenhagen (DK); Alexandre Gegelashvili, Thousand Oaks, CA (US); Yangkun Song, Thousand Oaks, CA (US); Thomas Peter Sonderby, Thousand Oaks, CA (US)

(21) Appl. No.: 18/696,203

(22) PCT Filed: Oct. 5, 2022
(86) PCT No.: PCT/US2022/045717

§ 371 Date: Mar. 27, 2024

Related U.S. Application Data
(60) Provisional application No. 63/252,949, filed on Oct. 6, 2021.

(51) Int. Cl. A61M 5/32

(52) U.S. Cl. CPC A61M 5/3243; A61M 5/3213

(57) ABSTRACT

A drug delivery device includes a housing having proximal and distal ends and a longitudinal axis extending therebetween, an injection assembly at least partially disposed within the housing at or near the proximal end, a shield slidably coupled with the housing, a drive assembly operably coupled with the injection assembly and the shield, and a cap. The injection assembly includes a needle or a cannula. The shield is positionable in an extended position in which at least a proximal end thereof extends a distance beyond the proximal end of the housing. The drive assembly is engageable to deliver a medicament via the injection assembly. The cap is removably coupled with at least one of the shield or the housing and is adapted to limit movement of the shield when coupled with the shield and/or the housing such that the drive assembly is restricted from delivering the medicament via the injection assembly.

### CROSS-REFERENCE TO RELATED APPLICATION

Priority is claimed to U.S. Provisional Patent Application No. 63/252,949, filed Oct. 6, 2021, the entire contents of which are hereby incorporated by reference herein.

### FIELD OF DISCLOSURE

The present disclosure generally relates to drug delivery devices, and, more particularly, to impact activated brake features for drug delivery devices.

### BACKGROUND

Drug delivery devices, such as injectors, are used to deliver liquid drugs to a patient. Upon activation, a drug delivery device will expel a drug stored within an internal reservoir through a needle, cannula, or other delivery member into the patient. Some drug delivery devices, such as pen-type autoinjectors, may be positioned adjacent to a patient's skin to deliver a drug via an injection needle or some other means over a period of time. The drug delivery device may be positioned near the tissue of the patient's abdomen, thigh, arm, or some other portion of the patient's body.

Some devices may have drawbacks. Specifically, users may be frightened by an exposed injection needle or feel they are inherently incapable of performing an injection. Because of aversions to exposed needles, as well as health and safety issues that may be involved, various types of injectors and other devices have been developed for concealing needles from the user and automating the injection task to assist the user in performing the injection, ensure reliable delivery of the medication and ensure patient safety.

Typically, three tasks may be performed when injecting a drug into a patient with a hypodermic syringe: 1) insertion of the needle into the patient; 2) injection of the drug from the syringe into the patient; and 3) withdrawal of the needle after the injection has been completed. Generally, shield-activated devices use manual needle insertion techniques whereby a user simultaneously inserts a needle and initiates dosing through the action of retracting a shield relative to the rest of the device. In these devices, the needle may automatically insert upon manually activating the device. Button-activated devices typically employ automated needle insertion mechanisms whereby the needle is inserted mechanically and the dosing mechanism release is automatically delayed until the correct device state is achieved. Any or all of these devices may use manual and/or automated withdrawal mechanisms to retract the needle, and typically rely on springs or other power sources to generate forces required to perform the tasks. Occasionally, a user may inadvertently mishandle or drop the device prior to use. In these circumstances, if the device is dropped in certain orientations, inertial forces may cause the internal components to move relative to each other, which may result in inadvertent premature device activation. Such a premature activation may result in some or all of the desired drug from actually being delivered to the user, which may be wasteful and potentially harmful to the user and/or others.

The present disclosure sets forth drug delivery devices embodying advantageous alternatives to existing drug delivery devices, and that may address one or more of the challenges or needs mentioned herein.

### SUMMARY

In accordance with a first aspect, a drug delivery device includes a housing having proximal and distal ends and a longitudinal axis extending therebetween, an injection assembly at least partially disposed within the housing at or near the proximal end, a shield slidably coupled with the housing, a drive assembly operably coupled with the injection assembly and the shield, and a cap. The injection assembly includes a needle or a cannula. The shield is positionable in an extended position in which at least a proximal end thereof extends a distance beyond the proximal end of the housing. The drive assembly is engageable to deliver a medicament via the injection assembly. The cap is removably coupled with at least one of the shield or the housing and is adapted to limit movement of the shield when coupled with the shield and/or the housing such that the drive assembly is restricted from delivering the medicament via the injection assembly.

In some examples, the shield may further be positionable in a retracted position in which the proximal end of the housing protrudes a distance beyond the proximal end of the shield. In such examples, moving the shield to the retracted position engages the drive assembly to deliver the medicament via the injection assembly.

In some approaches, the cap may include a locking tab, and the shield may include a detent. At least a portion of the locking tab of the cap may be positionable within the detent. In such approaches, positioning the portion of the locking tab within the detent may prevent the shield from moving to the retracted position. Further, in these and other examples, the housing may include a ramp that urges the locking tab into the detent. In some examples, upon removing the cap from the shield and the housing, a gap is formed between the housing and the shield to permit relative movement therebetween.

In any of these examples, the shield may further include a snap to secure the cap therewith.

In accordance with a second aspect, a drug delivery device includes a housing having proximal and distal ends and a longitudinal axis extending therebetween, an injection assembly at least partially disposed within the housing at or near the proximal end, a shield slidably coupled with the housing, a drive assembly operably coupled with the injection assembly and the shield, and a cap. The injection assembly includes a needle or a cannula. The shield is positionable in an extended position in which at least a proximal end thereof extends a distance beyond the proximal end of the housing. The drive assembly is engageable to deliver a medicament via the injection assembly. The housing, the shield, and the cap cooperate to form a braking mechanism to prevent the drive assembly from activating to deliver the medicament via the injection assembly.

In accordance with a third aspect, a drug delivery device having a braking mechanism includes a housing having proximal and distal ends, a longitudinal axis extending therebetween, and a ramp member positioned at or near the proximal end, a shield slidably coupled with the housing, and a cap removably coupled with at least one of the shield of the housing. The shield is movable between an extended position wherein at least a proximal end thereof extends a distance beyond the proximal end of the housing and a retracted position wherein the proximal end of the housing protrudes a distance beyond the proximal end of the shield. The shield further includes a detent disposed along a length thereof. The cap includes at least one locking tab. When the cap is coupled with at least one of the shield or the housing, upon the shield moving towards the retracted position, the ramp member of the housing urges the at least one locking tab into engagement with the detent of the shield to restrict the shield from moving to the retracted position.

### BRIEF DESCRIPTION OF THE DRAWINGS

The above needs are at least partially met through provision of the impact activated brake feature for drug delivery devices described in the following detailed description, particularly when studied in conjunction with the drawings, wherein:

FIG. 1 illustrates a perspective view of an example drug delivery device in accordance with various embodiments;

FIG. 2 illustrates a cross-sectional view of the example drug delivery device of FIG. 1 in accordance with various embodiments;

FIG. 3A illustrates a cross-sectional view of an example rear sub-assembly of the example drug delivery device of FIGS. 1 & 2 in accordance with various embodiments;

FIG. 3B illustrates a cross-sectional view of an example drug storage container of the example drug delivery device of FIGS. 1-3A in accordance with various embodiments;

FIG. 3C illustrates a cross-sectional view of an example front sub-assembly of the example drug delivery device of FIGS. 1-3B in accordance with various embodiments;

FIG. 4 illustrates a cross-sectional view of an example cap of the example drug delivery device of FIGS. 1-3C in accordance with various embodiments;

FIG. 5 illustrates a cross-sectional view of an example brake feature of the example drug delivery device of FIGS. 1-4 in a pre-activated state in accordance with various embodiments; and

FIG. 6 illustrates a cross-sectional view of an example brake feature of the example drug delivery device of FIGS. 1-5 in a braking state in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, a drug delivery device is provided that prevents premature activation of the device both during and after an inadvertent drop. The drug delivery device provided herein incorporates a braking mechanism that retains components used to activate the device in instances where drop-related forces may occur. When a falling device comes to a stop due to contact with a surface (e.g., the floor, a table, etc.), the internal components ordinarily would move due to these inertial forces. However, the components of the device that are responsible for device activation are prevented from traveling to the same extent, and by using this relative difference in movement, the braking mechanism may remove kinetic energy from the device. After the impact, the internal components will return to their default location and the device will function as intended.

Turning to the Figures, a drug delivery device 10 for delivering a drug, which may also be referred to herein as a medicament or drug product, is provided. The drug may be, but is not limited to, various biologicals such as peptides, peptibodies, or antibodies. The drug may be in a fluid or liquid form, although the disclosure is not limited to a particular state. In certain liquid formulations, the drug may have a viscosity between approximately (e.g., ±10%) 1-13 centipoise (cP), approximately (e.g., ±10%) 1-30cP, approximately (e.g., ±10%) 1-60cP, or other suitable viscosity profiles. Other examples are possible.

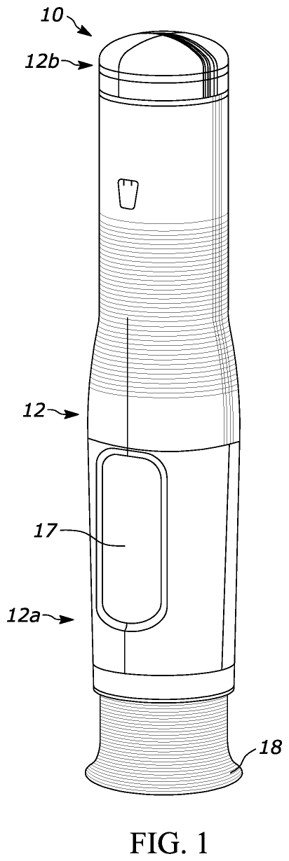

Various implementations and configurations of the drug delivery device 10 are possible. For example, while FIGS. 1-3C depict a first example device 10, the features of the described braking mechanism may be incorporated into any number of alternative delivery devices having different arrangements. The present drug delivery device 10 is in the form of a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). Further, in the illustrated examples, the drug delivery device 10 takes the form of an autoinjector or pen-type injector, and, as such, may be held in the hand of the user over the duration of drug delivery or dosing.

The configuration of various components included in the drug delivery device 10 may depend on the operational state of the drug delivery device 10. The drug delivery device 10 may have a pre-delivery or storage state, a delivery or dosing state, and a post-delivery state, although fewer or more states are possible. The pre-delivery state may correspond to the configuration of the drug delivery device 10 subsequent to assembly and prior to activation by the user. In some embodiments, the pre-delivery state may exist in the time between when the drug delivery device 10 leaves a manufacturing facility and when a patient or user activates a drive assembly of the drug delivery device 10. The delivery state may correspond to the configuration of the drug delivery device 10 while drug delivery is in progress. It is appreciated that during a transition from the pre-delivery state and the delivery state, the user may remove the drug delivery device 10 from any secondary packaging and begin positioning the drug delivery device 10 against the injection site. The post-delivery state may correspond to the configuration of the drug delivery device 10 after drug delivery is complete and/or when a stopper is arranged in an end-of-dose position in a drug storage container. For the purposes of the present disclosure, only the pre-delivery state and a portion of the delivery state will be described herein, as the braking mechanism described herein serves to maintain the drug delivery device 10 in the pre-delivery state in the event of accidental and/or unintended drops or contact.

The drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis A between a proximal end 12a and a distal end 12b. The drug delivery device 10 further includes an injection assembly 15 and a drive assembly 30. The injection assembly 15 and the drive assembly 30 may each be at least partially disposed within the housing 12. The injection assembly 15 includes a delivery member 16 in the form of a needle or a cannula. An opening 14 may be formed in the proximal end 12a to permit an insertion end 16a of the delivery member 16 to extend outside of (i.e., beyond the length of) the housing 12.

A transparent or semi-transparent inspection window 17 may be positioned in a wall of the housing 12 to permit a user to view component(s) inside the drug delivery device 10, including a drug storage container 23 (which is also part of the injection assembly 15). Viewing the drug storage container 23 through the window 17 may allow a user to confirm that drug delivery is in progress and/or complete. A removable cap 18 may cover the opening 14 prior to use of the drug delivery device 10, and, in some embodiments, may including a gripper 21a configured to assist with removing a sterile barrier 21 (e.g., a rigid needle shield (RNS), a flexible needle shield (FNS), etc.) mounted on the insertion end 16a of the delivery member 16. The gripper 21a may include one or more inwardly protruding barbs or arms that frictionally or otherwise mechanically engage the sterile barrier 21 to pull the sterile barrier 21 with the removable cap 18 when the user separates the removable cap 18 from the housing 12. Thus, removing the removable cap 18 has the effect of removing the sterile barrier 21 from the delivery member 16.

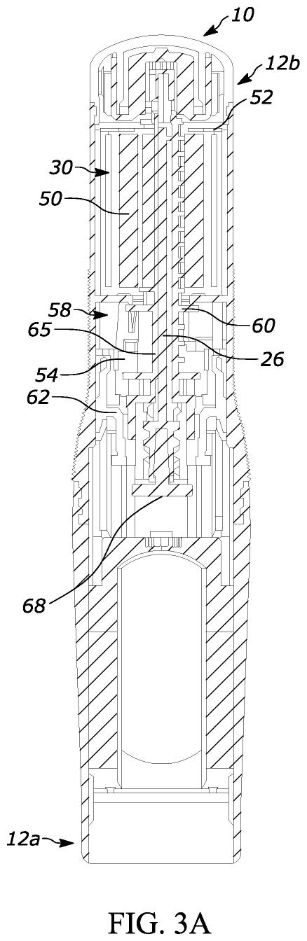

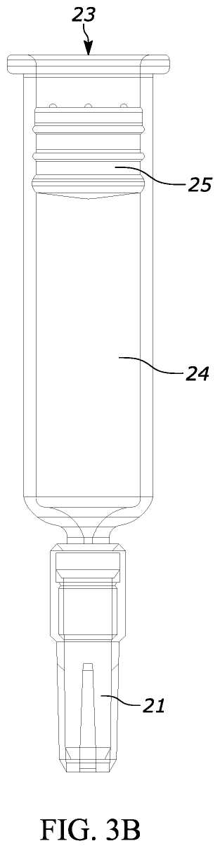

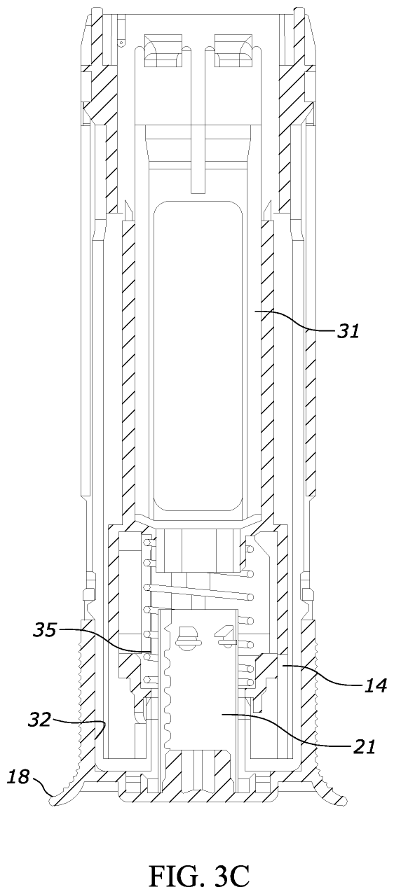

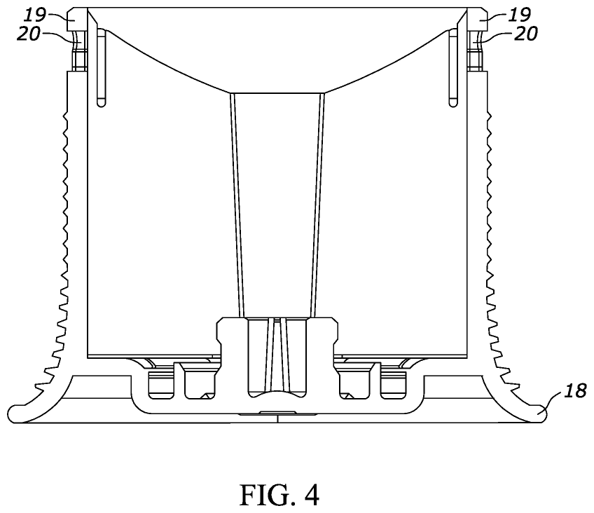

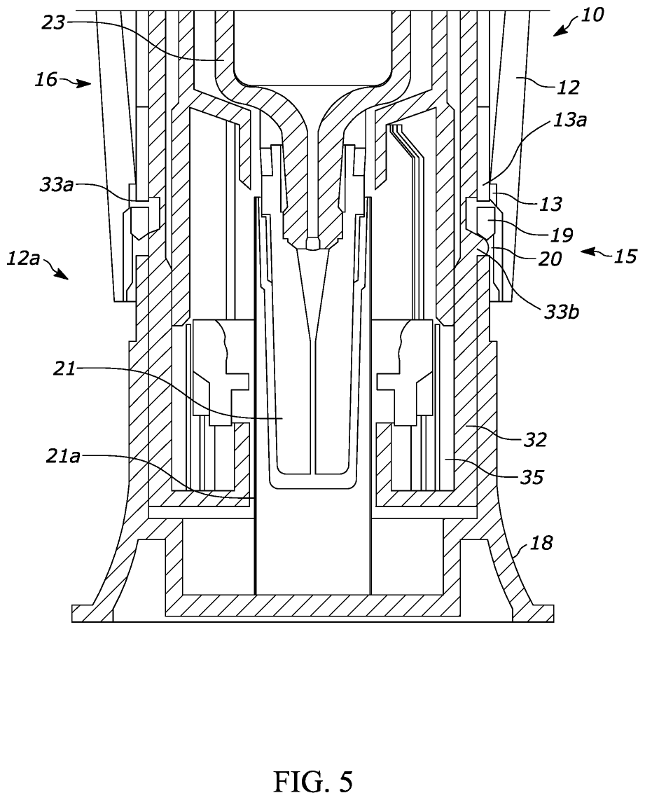

As illustrated in FIGS. 3-5, the cap 18 is in the form of a generally hollow member that includes any number of locking tabs 19 positioned at a distal end thereof. Further, the cap 18 includes an opening 20, which, in some examples, is positioned adjacent to the locking tab 19. The cap 18 may be removably coupled with the housing 12, and/or, as will be described in further detail below, a shield 32. More specifically, in the illustrated example of FIGS. 4 and 5, a portion of the cap 18 is insertable into the opening 14 formed by the housing 12.

With particular reference to FIGS. 4 and 5, an inner sidewall of the housing 12 includes any number of ramps 13 disposed at or near the proximal end 12a thereof. In the illustrated example, the housing 12 includes two ramps 12 spaced on opposing sides of the inner sidewall, though other arrangements are possible. The ramps 13 are in the form of tapered or angled protrusions that cause an inner diameter of the inner sidewall to decrease when progressing from the proximal end 12a of the housing towards the distal end 12b thereof. In some examples, the ramps 13 may be integrally formed with the housing 12. In other examples, the ramps 13 may be operably coupled with the housing 12 via any number of suitable approaches such as, for example, adhesives, fusion welding, a friction-fit coupling, and the like. Other examples are possible.

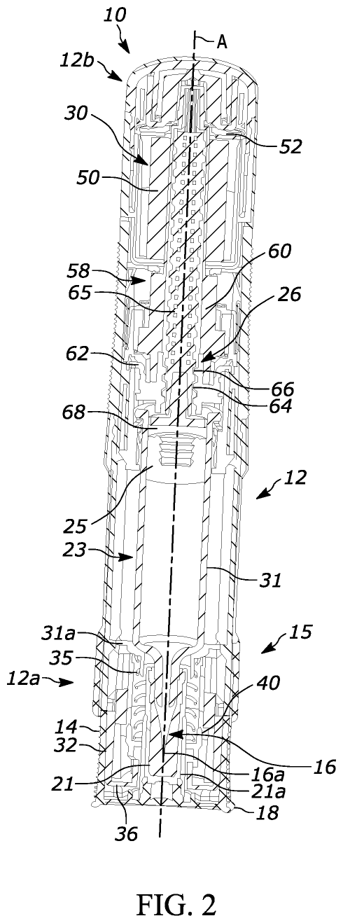

The housing 12 may have a hollow and generally cylindrical or tubular shape, and may include a rear cover having a generally hemispherical shape or a hollow cylindrical shape with an open end and a closed off end. In some embodiments, the housing, and any components to be contained therein, may be assembled together to define various sub-assemblies (e.g., a rear sub-assembly as illustrated in FIG. 2A and a front sub-assembly as illustrated in FIG. 2C). In some embodiments, the rear and front sub-assemblies are assembled independently of each other and then later combined with one another, as well as with the drug storage container 23, to form the fully-assembled drug delivery device 10. In certain such embodiments, some or all of the foregoing phases of assembly may occur in different manufacturing facilities or environments. In alternative embodiments, the housing 12 may be constructed in one piece, such that the housing 12 is defined by single, monolithic structure.

The drug storage container 23 is disposed within an interior space of the housing 12 and is configured to contain a drug 24. The drug storage container 23 may be pre-filled and shipped, e.g., by a manufacturer, to a location where the drug storage container 23 is combined with a remainder of the drug delivery device 10. The housing 12 may be pre-loaded with the drug storage container 23, e.g., by a manufacturer, or alternatively, loaded with the drug storage container 23 by a user prior to use of the drug delivery device 10. The drug storage container 23 may include a rigid wall defining an internal bore or reservoir. The wall may be made of glass or plastic. A stopper 25 may be moveably disposed in the drug storage container 23 such that it can move in an axial direction along the longitudinal axis A between the distal end and the proximal end the drug storage container 23. The stopper 25 may be constructed of rubber or any other suitable material. The stopper 25 may slidably and sealingly contact an interior surface of the wall of the drug storage container 23 such that the drug 24 is prevented or inhibited from leaking past the stopper 25 when the stopper 25 is in motion. Proximal movement of the stopper 25 expels the drug 24 from the reservoir of the drug storage container 23 into the delivery member 16.

The distal end of the drug storage container 23 may be open to allow a plunger 26 to extend into the drug storage container 23 and push the stopper 25 in the proximal direction. In the present embodiment, the plunger 26 and the stopper 25 are initially spaced from each other by a gap. Upon activation of a drive assembly 30, the plunger 26 moves in the proximal direction to close the gap and comes into contact with the stopper 25. Subsequent proximal movement of the plunger 26 drives the stopper 25 in the proximal direction. In alternative embodiments, the stopper 25 and the plunger 26 may be coupled to each other, e.g., via a threaded coupling, such that they move together jointly from the start of movement of the plunger 26. Once the stopper 25 is in motion, it may continue to move in the proximal direction until it contacts a distally-facing portion of the interior surface of the wall of the drug storage container 23. This position of the stopper 25 may be referred to as the end-of-dose position and may correspond to when delivery of the drug 24 to the patient is complete or substantially complete.

The delivery member 16 is connected or operable to be connected in fluid communication with the reservoir of the drug storage container 23. A proximal end of the delivery member 16 may define the insertion end 16a of the delivery member 16. The insertion end 16a may include a sharpened tip of other pointed geometry allowing the insertion end 16a to pierce the patient's skin and subcutaneous tissue during insertion of the delivery member 16. The delivery member 16 may be hollow and have an interior passageway. One or more openings may be formed in the insertion end 16a to allow drug to flow out of the delivery member 16 into the patient.

In the present embodiment, the drug storage container 23 is a pre-filled syringe and has a staked, hollow metal needle for the delivery member 16. Here, the needle is fixed relative to the wall of the drug storage container 23 and is in permanent fluid communication with the reservoir of the drug storage container 23. In other embodiments, the drug storage container 23 may be a needle-less cartridge, and, as such, initially may not be in fluid communication with the delivery member 16. In such embodiments, the drug storage container 23 may move toward a distal end of the delivery member 16, or vice versa, during operation of the drug delivery device 10 such that the distal end of the delivery member 16 penetrates through a septum covering an opening in the drug storage container 23 thereby establishing fluid communication with the reservoir of the drug storage container 23.

The drug storage container 23 may be fixed relative to the housing 12 such that the drug storage container 23 does not move relative to the housing 12 once installed in the housing 12. As such, the insertion end 16a of the delivery member 16 extends permanently through the opening 14 in the housing 12 in the pre-delivery, delivery, and post-delivery states. In the present embodiment, a container holder 31 fixes the position of the drug storage container 23 within the housing 12. The container holder 31 may have a hollow and generally cylindrical or tubular shape, and the drug storage container 23 may be disposed partially or entirely within the container holder 31. A proximal end of the container holder 31 may include an inwardly protruding flange 31a abutting against a neck of the drug storage container 23, thereby preventing proximal movement of the drug storage container 23. The container holder 31 may be fixedly attached to the housing 12 such that the container holder 31 is prevented from moving relative to the housing 12 during operation of the drug delivery device 10.

In alternative embodiments, the drug storage container 23 may be moveably coupled to the housing 12 such that the drug storage container 23 is able to move relative to the housing 12 during operation of the drug delivery device 10. In certain such alternative embodiments, the insertion end 16a of the delivery member 16 may be retracted within the opening 14 in the housing 12 in the pre-delivery state. Subsequently, during operation of the injection device 10, the insertion end 16a of the delivery member 16 may be deployed through the opening 14 in the housing 12 for insertion into the patient. This motion may, in some embodiments, be the result of the drug storage container 23 having been driven in the proximal direction relative to the housing 12.

The plunger 26 may be constructed in multiple, interconnected pieces, or alternatively, have a one-piece construction. In the present embodiment, the plunger 26 includes a rod 65 having a threaded outer surface 66 and washer or disk 68 rigidly attached to a proximal end of the rod 65. The disk 68 may impact and push the stopper 25 when the drive assembly 30 is activated. Accordingly, in some embodiments, the disk 68 may have shock-absorbing properties to attenuate any shock or vibrations associated with the impact event.

The drug delivery device 10 may further include a guard mechanism for preventing contact with the insertion end 16a of the delivery member 16 when the drug delivery device 10 is not being used to administer an injection. The guard mechanism may include a shield 32 moveably disposed at the proximal end 12a of the housing 12 adjacent to the opening 14. The shield 32 may have a hollow and generally cylindrical or tubular shape. The shield 32 may have a distal end received within the housing 12, and may be configured to move relative to the housing 12 between an extended position wherein a proximal end of the shield 32 extends through the opening 14 in the housing 12 and a retracted position wherein the proximal end of the shield 32 is retracted, fully or partially, into the opening 14 in the housing 12. In at least the extended position, the shield 32 may extend beyond and surround the insertion end 16a of the delivery member 16. In some embodiments, moving the shield 32 toward the retracted position may expose the insertion end 16a of the delivery member 16. Further, in some embodiments, the shield 32 may be coupled to the housing 12 and/or the container holder 31 via, for example, a pin-and-slot arrangement such that the shield 32 is able to translate in a linear direction relative to the housing 12 and/or the container holder 31 but is prevented from rotating relative to the housing 12 and/or the container holder 31.

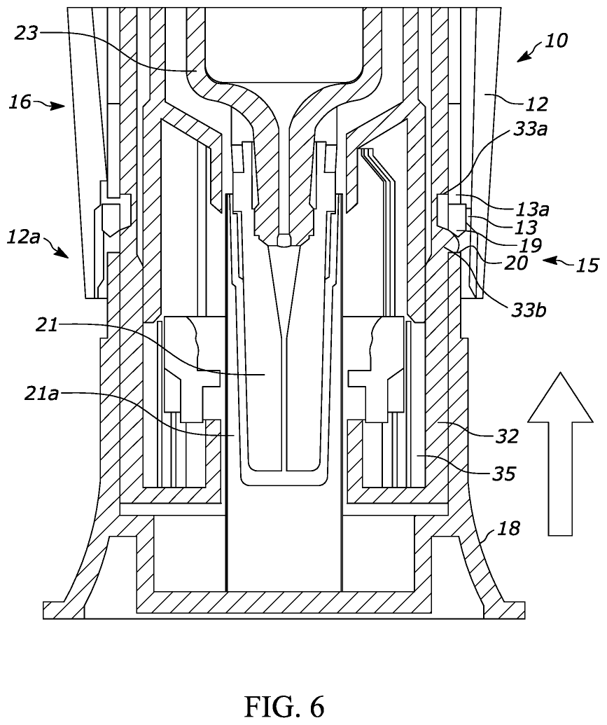

As illustrated in FIGS. 5 & 6, the shield 32 includes a detent 33a. The detent 33a is positioned along a length of the shield 32, and defines a groove or cavity portion that may extend around all or a portion of the exterior surface thereof. Further, in some examples, the shield 32 includes a snap 33b. Like the detent 33a, in some examples, the snap 33b is positioned along a length of the shield 32. The detent 33b defines a protrusion or ridge that may extend around all or a portion of the exterior surface of the shield 32. The snap 33b may be in the form of a member having an angled distal surface and a generally horizontal proximal surface when viewed in FIGS. 5 and 6. In the illustrated examples, the snap 33b is positioned adjacent to the detent 33a while being disposed in the proximal direction relative thereto. Other arrangements or configurations are possible.

The distal and proximal ends of the shield 32 may include, respectively, an activator portion 34 and a skin-contacting portion 36 (FIG. 3). In some embodiments, the detent 33a, the snap 33b, the activator portion 34, and the skin-contacting portion 36 may be integrally formed to define a single, monolithic structure. In other embodiments, the detent 33a, the snap 33b, the activator portion 34, and the skin-contacting portion 36 may be physically separate structures that are fixedly attached to each other such that they are immovable relative to each other and/or move jointly when in motion. At least the skin-contacting portion 36 of shield 32 may have a hollow and generally cylindrical or tubular shape and, in some embodiments, may be centered about the longitudinal axis A of the drug delivery device 10.

Moving the shield 32 from the extended position to the retracted position may be accomplished by pressing the skin-contacting portion 36 against the patient's skin at the injection site. In embodiments where the delivery member 16 protrudes from the opening 14 in the housing 12 in the pre-delivery or storage state, this motion may result in the insertion end 16a of the delivery member 16 being inserted into the patient's skin.

The guard mechanism may further include a guard biasing member 35. The guard biasing member 35 may bias or urge the guard 32 towards the extended position by exerting a biasing force in the proximal direction on the shield 32. In some examples (e.g., FIGS. 1-2C), the guard biasing member 35 is in the form of a compression spring. In other examples (e.g., FIGS. 4 & 5), the guard biasing member 35 may be in the form of a torsion or other form of spring. In any event, a user may overcome this biasing force by pressing the shield 32 against the injection site. When the injection is complete and the drug delivery device 10 is lifted off of the injection site, the guard biasing member 35 may return the shield 32 to the extended position, thereby covering the insertion end 16a of the deliver member 16. In some embodiments, the guard biasing member 35 may be positioned in the axial direction between, and in contact with both, a distally facing inner surface of the shield 32 and a proximally facing inner or outer surface of a lock 40. In embodiments where the shield 32 is a compression spring, movement of the shield 32 in the distal direction may cause the guard biasing member 35 to be compressed between the shield 32 and the lock 40. In some embodiments, the guard biasing member 35 may be partially compressed prior to retraction of the shield 32 and thus exert a biasing force on both the shield 32 and the lock 40 in the pre-delivery state.

As previously noted, the drug delivery device 10 may further include the drive assembly 30 disposed partially or entirely within the housing 12. Generally, the drive assembly 30 may be configured to store energy and, upon or in response to activation of the drive assembly 30 by the user, release or output that energy to drive the injection assembly 15 (i.e., the delivery member 16, the drug storage container 23, the stopper 25, and the plunger 26) to expel the drug 24 from the drug storage container 23 through the delivery member 16 into the patient. In the present example, the drive assembly 30 is configured to store mechanical potential energy; however, alternative embodiments of the drive assembly 30 may be configured differently, with, for example, the drive assembly 30 storing electrical or chemical potential energy. Upon activation of the drive assembly 30, the drive assembly 30 may convert the potential energy into kinetic energy for moving the plunger 26.

Generally, the drive assembly 30 may include a rotational biasing member 50, a rotational biasing member housing 52, a trigger ring 54, and a mechanical linkage 56. The rotational biasing member 50 may be a torsion spring (e.g., a spiral torsion, a helical torsion spring, etc.) which is initially retained in an energized state. In the energized state, the rotational biasing member 50 may be twisted or wound and retained in that twisted or wound configuration by the trigger ring 54 via the mechanical linkage 56. When released, the rotational biasing member 50 will try to return to its natural length or shape, and as a result, exert a biasing force causing the mechanical linkage 56 to rotate. The mechanical linkage 56, in turn, may convert the rotational motion into linear motion for driving the plunger 26 in the proximal direction. In some embodiments, the mechanical linkage 56 may convert the rotational motion from the rotational biasing member 50 into linear motion for driving the plunger 26 in the proximal direction and rotational motion of the plunger 26 about the longitudinal axis A.

Alternative embodiments may utilize an energy source different from a rotational biasing member. Certain alternative embodiments may utilize, for example, a linear biasing member (e.g., a helical compression spring, a helical extension spring, etc.) which, when released, outputs a force in the direction of travel of the plunger 26. In addition to or as an alternative to a biasing member, other embodiments may include any one or combination of: an electromechanical arrangement including an electric motor and/or solenoid and a drive train or transmission coupled to the plunger 26; or an arrangement that generates or releases a pressurized gas or fluid to propel the plunger 26 or which acts directly on the stopper 25 to move stopper 25 through the drug storage container 23 to expel the drug 24 from therein. In embodiments where the drug storage container 23 and/or the delivery member 16 is moveable relative to the housing 12, the drive assembly 30 may, upon activation, drive the drug storage container 23 and/or the delivery member 16 in the proximal direction so as to cause the insertion end 16a of the delivery member 16 to be inserted into the patient. Thus, in certain embodiments, the drive assembly 30 may provide the motive force needed for both inserting the delivery member 16 into the patient and expelling the drug 24 from the drug storage container 23.

The mechanical linkage 56 may include a plunger guide 60 and a nut 62. The plunger guide 60 may have a hollow and generally cylindrical or tubular shape. The distal end of the plunger 26 may be disposed inside of the plunger guide 60 in at least the pre-delivery state. A distal extend of the plunger guide 60 may extend through the center of the rotational biasing member 50 and may be coupled to the rotational biasing member 50 such that the plunger guide 60 rotates jointly together with the rotational biasing member 50 when the rotational biasing member 50 is released. An inner surface of the plunger guide 60 is coupled to an outer surface of the plunger 26 such that the plunger 26 rotates jointly together with plunger guide 60 when the rotational biasing member 50 is released, while permitting axial movement of the plunger 26 relative to the plunger guide 60. The coupling between the plunger guide 60 and the plunger 26 may be achieved via, for example, a splined arrangement, wherein a longitudinal protrusion on one of the inner surface of the plunger guide 60 or the outer surface of the plunger 26 is slidably received in a longitudinal slot on the other one of the outer surface of the plunger 26 or the inner surface of the plunger guide 60. The nut 62 may have a generally annular shape and may be disposed around a proximal end of the plunger 26 in the pre-delivery state. The nut 62 may be fixedly mounted such that the nut 62 is immoveable relative to the rear housing 27. Furthermore, the nut 62 may have a threaded inner surface 64 which engages the threaded outer surface 66 of the plunger 26. As a consequence of this threaded engagement, rotation of the plunger 26 relative to the nut 62 may drive the plunger 26 linearly in the proximal direction. This in turn causes the plunger 26 to act on and push the stopper in the proximal direction to expel the drug 24 from the storage container 23 into the patient via the inserted delivery member 16.

The shield 32 may be configured to interact with the drive assembly 30 when the shield 32 moves from the extended position to the retracted position. This interaction may activate the drive assembly 30 to output the energy needed for driving the plunger 26 to expel the drug 24 from the drug storage container 23 and/or insert the insertion end 16a of the delivery member 16 into the patient's skin. In the present embodiment, movement of the shield 32 from the extended position to the retracted position releases the rotational biasing member 50 from the energized state, thereby allowing the rotational biasing member 50 to de-energize and drive the plunger 26, via the mechanical linkage 56, to expel the drug 24 from the drug storage container 23. More particularly, in the pre-delivery state, the trigger ring 54 may be arranged in an initial position where it lockingly engage an exterior surface of the plunger guide 60, thereby preventing the plunger guide 60 from rotating under the biasing force of the rotational biasing member 50. As a consequence, the rotational biasing member 50 is prevented de-energizing. When the shield 32 moves from the extended position to the retracted position as a result of being pressed against the patient's skin, the activator portion 34 of the shield 32 pushes the trigger ring 54 in the distal direction to a releasing position where the trigger ring 54 disengages from the plunger guide 60. As a consequence, the plunger guide 60 is able to rotate under the biasing force of the rotational biasing member 50 and drive, via the threaded connection between the plunger 26 and the nut 62, the plunger 26 in the proximal direction.

In an alternative embodiment, the trigger ring 54 may be omitted, and the activator portion 34 of the shield 32 may, when the shield 32 is in the extended position, lockingly engage the exterior surface of the plunger guide 60 to prevent it from rotating. Thus, the shield 32 may retain the rotational biasing member 50 in the energized state in such an embodiment. When the shield 32 moves from the extended position to the retracted position, the activator portion 34 of the shield 32 may disengage from the plunger guide 60, thereby freeing the plunger guide 60 to rotate under the biasing force of the biasing member 50.

The rotational biasing member housing 52 may be disposed within and rigidly attached to the housing 12. The rotational biasing member housing 52 may have a hollow and generally cylindrical or tubular shape, and may receive, in full or in part, the rotational biasing member 52 such that the rotational biasing member housing 52 surrounds or partially surrounds the rotational biasing member 50. The rotational biasing member housing 52 may serve as a mount or seat for the rotational biasing member 50 to push off of when released.

Having described the general configuration and operation of the drug delivery device 10, it is appreciated that axial movement of the shield 32 towards the distal end 12b of the housing 12 serves to activate the drive assembly 30 to deliver the drug 24 via the injection assembly 15. However, it may be the case that at some point during the pre-activated state, a user may inadvertently drop or jostle the drug delivery device 10 in a way that the cap 18, when coupled with the device 10, may urge the shield 32 towards the distal end 12b of the housing 12. As illustrated in FIGS. 4 and 5, in the pre-activated state, the cap 18 at least partially surrounds a portion of the shield 32 while being at least partially disposed within the proximal end 12a of the housing 12. More specifically, in this configuration, the cap 18 may be removably coupled with the housing 12 and/or the shield 32. In this pre-activated state, the snap 33b of the shield 32 is positioned within the opening 20 formed on the cap 18 to maintain the coupling of the cap 18 and the housing 12 and/or the shield 32. In this arrangement, to prepare for drug administration, a user would pull the cap 18 in the proximal direction 12a away from the device 10 to uncouple the opening 20 and the snap 33b. Notably, in this arrangement, the ramps 13 of the housing 12 are positioned adjacent to the locking tabs 19 formed on the cap 18.

Prior to a user deciding to proceed with drug administration, the housing 12, the cap 18, and the shield 32 cooperate to form a braking mechanism that prevents the drive assembly 30 from activating. More specifically, if the cap 18 is coupled with the housing 12 and/or the shield 32, the interconnection of these components prevents the shield 32 from moving axially in the distal direction. With reference to FIG. 6, in the event of an accidental drop and/or jostling of the device 10, inertial and/or contact forces may cause the cap 18 to move in the axial direction towards the distal end 12b of the housing 12. Upon such movement occurring, the locking tabs 19 engage and slide along the ramps 13, which urge the locking tabs 19 inwards in a radial direction towards the longitudinal axis A. This relative movement causes the locking tabs 19 to engage the detents 33a and be at least partially inserted therein, and as such, the locking tabs 19 restrict or prevent the shield 32 from further advancing axially towards the distal end 12b of the housing. In some examples, the locking tabs 19 may additionally engage a ledge 13a that may assist with retaining and positioning the locking tabs 19 within the detent 32a.

After a period of time, the internal components will stop moving relative to each other, returning to their default location, and the cap 18 and the locking tabs 19 will disengage from the detents 33a of the shield 32, thereby permitting the device 10 to be used as desired (i.e., remaining in the pre-activated state and/or removing the cap 18 to transition to the delivery or dosing state. Here,

The user may pull and detach the removable cap 18 from the housing 12 with sufficient force to overcome the retaining force between the opening 20 and the snap 33b. As a result of this motion, the gripper 21a may pull and detach the sterile barrier 21 from the drug storage container 23. This may uncover the insertion end 16a of the delivery member 16. Nevertheless, the insertion end 16a of the delivery member 16 will remain surrounded by the shield 32 at this stage. The user may position the skin-contacting portion 36 of the shield 32 over the desired injection site and then push the skin-contacting portion 36 against the injection site. The force applied by the user will overcome the biasing force of the guard biasing member 35, thereby causing the shield 32 to retract into the opening 14 moving from the extended position to the retracted position in the distal direction. Notably, when the cap 18 is removed from the device 10, a gap is formed between the ramps 13 formed on the housing 12 and the shield 32, and as such, the ramps 13 do not contact or otherwise engage the shield 32, thereby permitting relative movement therebetween. The delivery member 16 remains stationary relative to the housing 12 during the retracting movement of the shield 32.

The retraction of the shield 32 may cause any number of actions to occur. Because the delivery member 16 remains stationary relative to the housing 12 during retraction of the shield 32, the insertion end 16a of the delivery member 16 is caused to protrude through an opening in the skin-contacting portion 36 of the shield 32 and thereby pierce the patient's skin at the injection site and penetrate into the patient's subcutaneous tissue. Retraction of the shield 32 may also activate the drive assembly 30. More particularly, retraction of the shield 32 may cause the activator portion 34 to engage the trigger ring 54 in the distal direction to the releasing position where the trigger ring 54 disengages from the plunger guide 60, thereby activating the drive assembly 30 to deliver the drug 24 via the injection assembly 15.

So configured, the braking mechanism serves to prevent inadvertent activation of the device if it is dropped. The angled ramps 13 inside the housing 12 urge the locking tabs 19 of the cap inwards and clamp onto the shield 32 in the event the cap 18 and the shield 32 travel excessively far into the device 10 in the distal direction. Such a braking mechanism removes energy from the shield to allow the device to return to its default, pre-activated state.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon@ (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-a4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin@ (somatropin, Human Growth Hormone); Herceptin@ (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret@ (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFa monoclonal antibody); Reopro® (abciximab, anti-GP Ilb/llia receptor monoclonal antibody); Actemra® (anti-IL6 Receptor mAb); Avastin® (bevacizumab), HuMax-CD4 (zanolimumab); MvasiTM (bevacizumab-awwb); Rituxan® (rituximab, anti-CD20 mAb); Tarceva® (erlotinib); Roferon-A@-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-a4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1receptor components (the Type I receptor and receptor accessory protein)); VEGF trap (lg domains of VEGFR1 fused to lgG1Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFa mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200(volociximab, anti-a5B1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNa mAb (MEDI-545, MDX-198); anti-IGF1R mAb; anti-IGF-1R mAb (HuMax-Inflam); anti-IL 12 mAb (ABT-874); anti-IL 12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGB mAb (MDX-1307); anti-mesothelin dsFv-PE38 conjugate (CAT-5001); anti-PD1mAb (MDX-1106 (ONO-4538); anti-PDGFRa antibody (IMC-3G3); anti-TGFß mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (lgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with AvsolaTM (infliximab-axxq), anti-TNF a monoclonal antibody, biosimilar to Remicade@ (infliximab) (Janssen Biotech, Inc.) or another product containing infliximab for the treatment of autoimmune diseases. In some embodiments, the drug delivery device may contain or be used with Kyprolis® (carfilzomib), (2S)-N- ((S)-1- ((S)-4-methyl-1- ((R)-2-methyloxiran-2-yl)-1-oxopentan-2-ylcarbamoyl)-2-phenylethyl)-2-((S)-2-(2- morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2- [(1S)-1-(3-ethoxy-4-methoxyphenyl)-2-(methylsulfonyl) ethyl]-2,3-dihydro-1,3-dioxo-1H-isoindol-4-yl] acetamide, or another product containing apremilast for the treatment of various inflammatory diseases. In some embodiments, the drug delivery device may contain or be used with ParsabivTM (etelcalcetide HCl, KAI-4169) or another product containing etelcalcetide HCI for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan@/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of lgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein (a), also known as Lp (a), or another product containing a small interfering RNA (siRNA) that lowers lipoprotein (a). In some embodiments, the drug delivery device may contain or be used with ABP 654 (human lgG1 kappa antibody), a biosimilar candidate to Stelara®, or another product that contains human lgG1 kappa antibody and/or binds to the p40 subunit of human cytokines interleukin (IL)-12 and IL-23. In some embodiments, the drug delivery device may contain or be used with AmjevitaTM or AmgevitaTM (formerly ABP 501) (mab anti-TNF human lgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)'anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin@ biologic under investigation as a treatment for solid tumors. In some embodiments, the drug delivery device may contain or be used with AMG 509 or another product containing a bivalent T-cell engager and is designed using XmAb@ 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vill (EGFRvlll) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing having a proximal end, a distal end, and a longitudinal axis extending between the proximal end and the distal end thereof;
an injection assembly at least partially disposed within the housing at or near the proximal end thereof, the injection assembly including a needle or a cannula;
a shield slidably coupled with the housing, the shield being positionable in an extended position in which at least a proximal end of the shield extends a distance beyond the proximal end of the housing;
a drive assembly at least partially disposed within the housing and operably coupled with the injection assembly and the shield, the drive assembly engageable to deliver a medicament via the injection assembly; and
a cap removably coupled with at least one of the shield or the housing;
wherein the cap is adapted to limit movement of the shield when coupled with the shield and/or the housing such that the drive assembly is restricted from delivering the medicament via the injection assembly.

2. The device of claim 1, wherein the shield is further positionable in a retracted position in which the proximal end of the housing protrudes a distance beyond the proximal end of the shield, wherein moving the shield to the retracted position engages the drive assembly to deliver the medicament via the injection assembly.

3. The device of claim 1, wherein the cap includes a locking tab and the shield includes a detent, at least a portion of the locking tab of the cap adapted to be positionable within the detent of the shield.

4. The device of claim 3, wherein positioning the portion of the locking tab within the detent prevents the shield from moving to the retracted position.

5. The device of claim 3, wherein the housing includes a ramp adapted to urge the locking tab into the detent.

6. The device of claim 5, wherein upon removing the cap from the shield and the housing, a gap is formed between the housing and the shield to permit relative movement therebetween.

7. The device of claim 1, wherein the shield further includes a snap to secure the cap therewith.

8. A drug delivery device comprising:
a housing having a proximal end, a distal end, and a longitudinal axis extending between the proximal end and the distal end thereof;
an injection assembly at least partially disposed within the housing at or near the proximal end thereof, the injection assembly including a needle or a cannula;
a shield slidably coupled with the housing, the shield being positionable in an extended position in which at least a proximal end of the shield extends a distance beyond the proximal end of the housing;
a drive assembly at least partially disposed within the housing and operably coupled with the injection assembly and the shield, the drive assembly engageable to deliver a medicament via the injection assembly; and
a cap removably coupled with at least one of the shield or the housing;
wherein the housing, the shield, and the cap cooperate to form a braking mechanism to prevent the drive assembly from activating to deliver the medicament via the injection assembly.

9. The drug delivery device of claim 8, wherein the braking mechanism includes:
a ramp member formed at or near the proximal end of the housing;
a detent disposed along a length of the shield; and
a locking tab formed on a portion of the cap;
wherein when the cap is coupled with at least one of the shield or the housing, upon the shield moving towards the retracted position, the ramp member of the housing urges the at least one locking tab into engagement with the detent of the shield to restrict the shield from moving to the retracted position.

10. The device of claim 9, wherein upon removing the cap from the shield and the housing, a gap is formed between the housing and the shield to permit relative movement therebetween.

11. The device of claim 8, wherein the shield further includes a snap to secure the cap therewith.

12. A drug delivery device having a braking mechanism, the drug delivery device comprising:
a housing having a proximal end, a distal end, a longitudinal axis extending between the proximal end and the distal end thereof, and a ramp member positioned at or near the proximal end;
a shield slidably coupled with the housing, the shield being movable between an extended position wherein at least a proximal end of the shield extends a distance beyond the proximal end of the housing and a retracted position wherein the proximal end of the housing protrudes a distance beyond the proximal end of the shield, the shield including a detent disposed along a length thereof;
a cap removably coupled with at least one of the shield or the housing, the cap including at least one locking tab;
wherein when the cap is coupled with at least one of the shield or the housing, upon the shield moving towards the retracted position, the ramp member of the housing urges the at least one locking tab into engagement with the detent of the shield to restrict the shield from moving to the retracted position.

13. The drug delivery device of claim 12, wherein upon removing the cap from the shield and the housing, a gap is formed between the ramp and the shield to permit relative movement therebetween.

14. The drug delivery device of claim 12, further comprising a drive assembly and an injection assembly, wherein moving the shield to the retracted position engages the drive assembly to deliver a medicament via the injection assembly.

15. The drug delivery device of claim 12, wherein the shield further includes a snap to secure the cap therewith.
